(19) United States
(12) Patent Application Publication
(10) Pub. No.: US 2022/0079421 A1
(43) Pub. Date: Mar. 17, 2022

(54) ENDOSCOPE FOR PERFORMING A RETROFLEXION MANOEUVRE

(71) Applicant: AMBU A/S, Ballerup (DK)

(72) Inventor: Jesper Domino Rask, København (DK)

(21) Appl. No.: 17/243,266

(22) Filed: Apr. 28, 2021

(30) Foreign Application Priority Data
Sep. 15, 2020 (DK) PA2020 70592

(51) Int. Cl. A61B 1/005; A61B 1/05; A61B 1/06; A61B 1/00

(52) U.S. Cl. CPC A61B 1/0057; A61B 1/0052; A61B 1/00045; A61B 1/0684; A61B 1/051

(57) ABSTRACT

An endoscope capable of performing a retroflexion manoeuvre, including a handle having a control mechanism, a distal tip part having a vision device with a field of view, a bending section having a first state, a retroflexion state, an insertion tube including an exterior surface facing the surroundings of the endoscope, and at least one steering wire connecting the control mechanism with the bending section so that manipulation of the control mechanism causes the endoscope to perform a retroflexion manoeuvre by bending the bending section from the first state to the retroflexion state, wherein the exterior surface of the insertion tube includes a coloured area of a colour configured to reduce over- or underexposure of the vision device in the retroflexion state.

### CROSS-REFERENCE TO RELATED APPLICATIONS

This application claims the benefit of and priority from Danish Patent Application No. PA 2020 70592, filed Sep. 15, 2020, which is incorporated herein by reference in its entirety.

### TECHNICAL FIELD

The present disclosure relates to an endoscope for performing a retroflexion manoeuvre and a method of making such an endoscope.

### BACKGROUND

Insertion endoscopes are well-known devices in the medical field for visually examining the interior of a hollow organ or cavity of a body, such as during urology procedures or gastrointestinal procedures, by means of inserting an insertion portion of the endoscope. The insertion portion of the endoscope comprises an elongated insertion tube, a distal tip part, and a bending section connecting the insertion tube with the distal tip part. The endoscope typically has a handle connected to the insertion tube and positioned at the proximal end of the endoscope as seen from the operator. The endoscope further has a vision device, such as a built-in camera or fibre optics. The vision device is typically incorporated in the distal tip part at the distal end of the endoscope. This definition of proximal as being closest to an operator and distal as being furthest from an operator is used throughout this disclosure. Illumination of the area in front of the distal tip part of the endoscope is normally required, in particular the field of vision of the vision device. One known way of achieving such illumination is to incorporate one or more Light Emitting Diodes (LEDs) in the distal tip part of the endoscope, e.g. as mentioned in WO2014/106511 disclosing a disposable endoscope. Alternatively, illumination may be provided by light guides and/or fibre optics guiding light from a light source outside the endoscope and to the distal tip part.

The bending section is provided in order to manoeuvre the endoscope inside the body cavity. The bending section has increased flexibility, e.g. achieved by a number of articulated segments of which the distal tip part forms the distalmost segment. Bending or straightening of the bending section in the insertion part of the endoscope is typically done by tensioning or slacking, respectively, steering wires running from the distal tip part through the remainder of articulated segments and along the inside of the elongated insertion tube to a control mechanism, such as a control lever, of the handle.

Data and/or power cables for the vision device (when being a camera) and other electronics, such as LED lighting accommodated in the distal tip part, also run along the inside of the elongated insertion tube and the bending section. Optionally, fibre optics/light guides for the illumination means, also run along the inside of the elongated insertion tube and the bending section from the handle to the distal tip part. Furthermore, a working channel may run along the inside of the insertion tube and the bending section from the handle to the tip part, e.g. allowing liquid to be removed from the body cavity or allowing the insertion of medical tools or surgical instruments into the body cavity.

In some procedures, a so-called retroflexion manoeuvre is performed in which the bending section bends backwards to allow the camera to inspect the course of the insertion tube and/or the anatomy adjacent to the insertion tube. One example is a urologic procedure, wherein the endoscope is inserted through the urethra and retroflexion is performed to inspect the area surrounding the urethra. Another example is a gastrointestinal procedure where it may also be necessary to perform inspection backwards by a retroflexion manoeuvre. During the retroflexion manoeuvre, the images produced by the vision device are often either over- or underexposed. When the images are overexposed, details are lost in the shadows and the darkest areas of the image. On the other hand, when the images are underexposed, details are lost in the highlights and the brightest parts of the image. Both cases can lead to poor image quality of portions of the image indicative of the anatomy of interest which makes it difficult for the operator to perform the inspection.

### SUMMARY

In light of the above, it may be seen as an object of the present disclosure to provide an endoscope with improved image quality during a retroflexion manoeuvre. Another object of the present disclosure is to provide a method of providing an endoscope with improved image quality during a retroflexion manoeuvre.

One or more of these objects may be met by aspects of the present disclosure as described in the following.

A first aspect of this disclosure relates to an endoscope capable of performing a retroflexion manoeuvre, comprising:
a handle having a control mechanism,
a distal tip part having a vision device, such as a camera, with a field of view, and an illumination device, such as one or more light emitting diodes (LEDs), for providing illumination for the vision device,
a bending section having a first state, a retroflexion state, and a distal end connected to the distal tip part,
an insertion tube extending from the handle to a proximal end of the bending section and including an exterior surface facing the surroundings of the endoscope, and
at least one steering wire connecting the control mechanism with the bending section so that manipulation of the control mechanism causes the endoscope to perform a retroflexion manoeuvre by bending the bending section from the first state to the retroflexion state,
   wherein the exterior surface of the insertion tube includes a coloured area of a colour configured to reduce over- or underexposure of the vision device when the endoscope is in the retroflexion state.

By marking at least a portion of the area of the exterior surface of the insertion tube that is visible and illuminated during retroflexion with an optimised colour, the coloured area will appear neither too dark nor too bright in images produced by the vision device, thus allowing the vision device to optimise the exposure level and thereby provide the operator with an improved image quality at least of the portion of the image not covered by the coloured area, i.e. the portion of the image indicative of the tissue or anatomy.

Additionally or alternatively, the first state may be a resting state of the endoscope in which the bending section is preferably substantially straight.

Additionally or alternatively, the coloured area may be visible within the field of view of the camera in the retroflexion state of the bending section.

Additionally or alternatively, the field of view of the vision device may include a view cone with a view cone angle (α_{col}) of at least 10°, 20°, 30°, 40°, or 50° encompassing the coloured area within the field of view when the bending section of the endoscope is in the retroflexion state. The view cone is a subset of the field of view so the maximum view cone angle equals the field of view angle.

This may provide the advantage that when the bending section of the endoscope is in the retroflexion state, the majority of the insertion tube within the field of view of the vision device, in particular the part of the insertion tube closest to the vision device, is occupied by the coloured area instead of the non-coloured remainder of the insertion tube. This may have the advantage of positioning the coloured area where the improvement of the exposure level is greatest.

Additionally or alternatively, the colour of the coloured area may be different from a colour of a majority of the insertion tube.

By only marking the coloured area of the first section that is visible during retroflexion with an optimised colour, an improved image quality is provided while the colour of the majority of the insertion tube can be left the designers preference.

Additionally or alternatively, the colour of the coloured area may be a grey colour, e.g. a colour with L* between 15 to 85, preferably L* between 30 to 70, more preferably L* between 40 to 60, as measured by a CIE L*a*b* colour code system. More preferably, the colour may be a grey colour with a* between negative 10 to positive 10 and b* between negative 10 to positive 10.

Such a colour may have the advantage of improving the image quality when the bending section of the endoscope is in the retroflexion state by reducing over- or underexposure of the vision device.

Additionally or alternatively, a first section of the exterior surface of the insertion tube may be visible within the field of view of the vision device when the bending section of the endoscope is in the retroflexion state. The coloured area may form part of the first section and preferably covers at least 20%, 40%, 60%, 80%, 90%, or 100% of the area of the first section. Additionally or alternatively, the coloured area may cover up to 100%, 90%, 80%, 70%, 60%, or 40% of the area of the first section. In particular, the coloured area may cover an area in the range of 20% to 80% of the area of the first section, preferably 40% to 80%, more preferably 60% to 80%.

By providing the coloured area on the visible first section, the exposure level of the vision device may be further improved. The exposure level is further improved by increasing the coverage of the coloured area on the first section.

Additionally or alternatively, the first section may have a proximal portion and a distal portion. The coloured area may cover the distal portion of the first section. The distal portion may have a longitudinal extent of at most 90%, 80%, 70%, 60%, 50%, 40%, 30%, or 20% relative to a longitudinal extent of the exterior surface of the insertion tube. Additionally or alternatively, the coloured area may not cover the proximal portion of the first section.

By providing the coloured area on a distal portion of the insertion tube, the image quality can be improved while minimising the extent of the coloured area.

Additionally or alternatively, the exterior surface of the insertion tube may have a proximal portion and a distal portion. The coloured area may cover a distal portion of the the insertion tube having an extent of at most 90%, 80%, 70%, 60%, 50%, 40%, 30%, or 20% relative to a longitudinal extent of the exterior surface. Additionally or alternatively, the coloured area may not cover the proximal portion of the exterior surface.

Additionally or alternatively, a bending angle of the bending section between the first state and the retroflexion state may be at least 180 degrees, preferably at least 210 degrees.

Additionally or alternatively, the insertion tube may comprise a plurality of depth marks, preferably spaced, optionally equidistantly, along the extent of the insertion tube. The depth marks may be of the same colour as the coloured area.

Such depth marks may allow the operator to easily infer how far the insertion tube has been inserted into a patient.

Additionally or alternatively, the endoscope may be a cystoscope, a ureteroscope or an endoscope used during urology procedures. Alternatively, the endoscope may be configured for use in gastrointestinal procedures, such as a duodenoscope, a gastroscope, a colonoscope.

Additionally or alternatively, the endoscope may be a disposable endoscope which may not be intended to be cleaned and/or sterilised for reuse.

A second aspect of this disclosure relates to an endoscope vision system comprising an endoscope according to the first aspect of this disclosure and a monitor, and wherein the endoscope is connectable to the monitor via one or more cables or via a wireless connection, e.g. a standard radiofrequency wireless connections such as Bluetooth, Wi-Fi, etc. The monitor includes a display unit configured for displaying images captured by the vision device of the endoscope.

A third aspect of this disclosure relates to a method for marking an area of an endoscope part for an endoscope capable of performing a retroflexion manoeuvre, the endoscope comprising:

a handle having a control mechanism;

a distal tip part having a vision device, such as a camera, with a field of view, and an illumination device, such as one or more light emitting diodes (LEDs), for providing illumination for the vision device;

a bending section having a first state, a retroflexion state;

a distal end connected to the distal tip part;

an insertion tube extending from the handle to a proximal end of the bending section and including an exterior surface facing the surroundings of the endoscope; and

at least one steering wire connecting the control mechanism with the bending section so that manipulation of the control mechanism causes the endoscope to perform a retroflexion manoeuvre by bending the bending section from the first state to the retroflexion state;

wherein the method comprises the steps of:
providing the insertion tube configured for insertion into a patient and including the exterior surface facing the surroundings, and
locating an area of the exterior surface of the insertion tube being visible within a field of view of the vision device of the distal tip part of the endoscope when the insertion tube forms part of the endoscope and the bending section is in the retroflexion state, and
marking the area in a colour configured to reduce over- or underexposure of the vision device of the distal tip part of the endoscope thereby providing a coloured area on exterior surface of the insertion tube.

By marking the area that is visible during retroflexion with an optimised colour, the vision device may be able to provide the operator with an improved image quality.

Additionally or alternatively, the method comprises a step of assembling the endoscope part with the remaining parts of the endoscope to obtain the endoscope.

Additionally or alternatively, the insertion tube comprises one or more additives configured for being activated in a laser marking process.

This may allow the provision different shadings and/or colourings to the coloured area, e.g. providing a gradual colouring.

Additionally or alternatively, the coloured area may be marked by a laser in a laser marking process.

Additionally or alternatively, the step of marking the area may comprise causing the laser to at least partially melt the exterior surface of the insertion tube within the area during the laser marking process. This may reduce the reflectivity of the area and thus reduce over- or underexposure.

It has been have found that over- or underexposure of the vision device is likely caused by a combination of the colour and the reflectivity of the exterior surface. By marking the coloured area using a laser marking process may also reduce the reflectivity of the surface, thus further allowing the vision device to optimise the exposure level to prevent loss of image detail. Only a slight laser treatment may be required to significantly reduce the reflectivity of the exterior surface.

Additionally or alternatively, the method may further comprise a step of marking two or more depth marks, optionally equidistantly, along the extent of the insertion tube.

Additionally or alternatively, the method may comprise a step of marking a quality inspection mark on the insertion tube indicating that the endoscope has passed quality inspection.

Additionally or alternatively, the coloured area, the depth marks, and optionally the quality inspection mark may be marked using the same marking process, preferably the same laser marking process, optionally either sequentially by using the same laser or simultaneously by using different lasers.

Such a method may improve the manufacturing efficiency since the coloured area, the depth marks, and optionally the quality inspection mark can be provided in the same process.

The features discussed in the present disclosure are particularly applicable to single use endoscopes, which are disposable and not intended to be cleaned and reused. The features discussed in the present disclosure may, however, also be applied to reusable endoscopes.

A person skilled in the art will appreciate that any one or more of the above aspects of this disclosure and embodiments thereof may be combined with any one or more of the other aspects of this disclosure and embodiments thereof.

### BRIEF DESCRIPTION OF THE DRAWINGS

Embodiments of this disclosure will be described in more detail in the following with regard to the accompanying figures. The figures show one way of implementing the present disclosure and are not to be construed as being limiting to other possible embodiments falling within the scope of the attached claim set.

FIG. 1 is a schematic perspective illustration of an endoscope according to this disclosure,

FIG. 2 is a schematic perspective illustration of a monitor connectable to the endoscope,

FIG. 3 is a schematic illustration of the endoscope in a retroflexion state, and

FIG. 4 is a schematic illustration of the distal end of the insertion tube in the retroflexion state.

### DETAILED DESCRIPTION

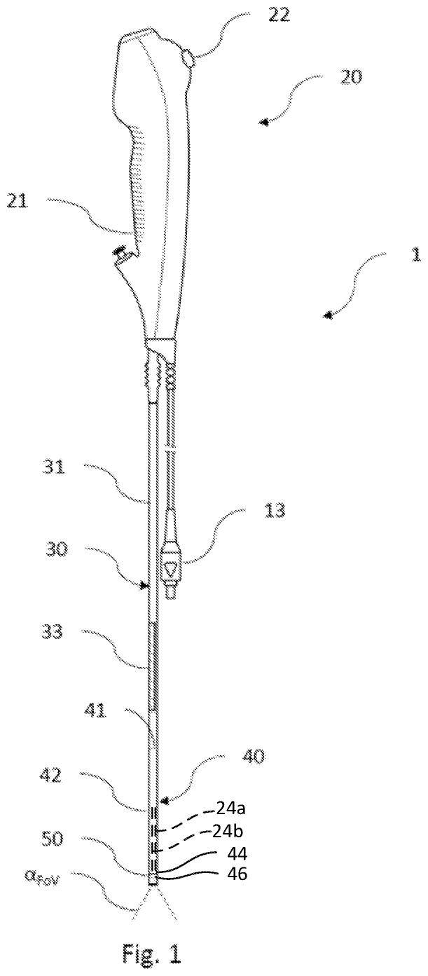

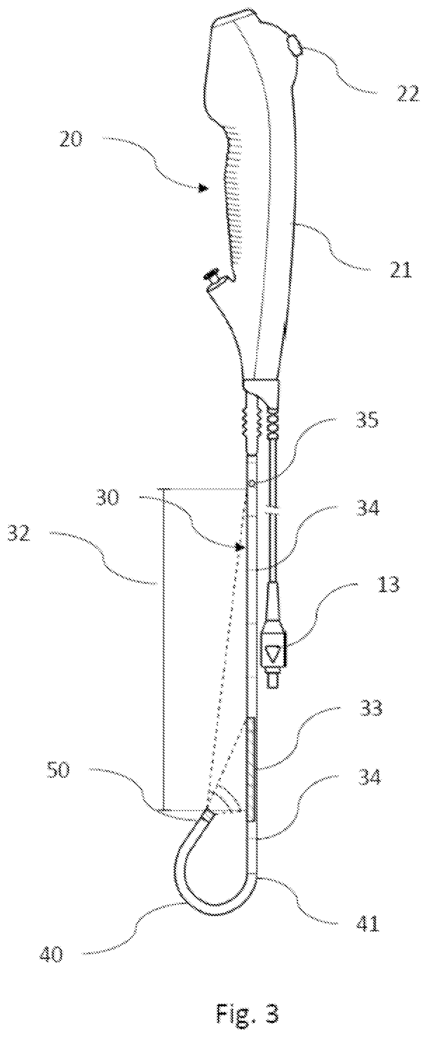

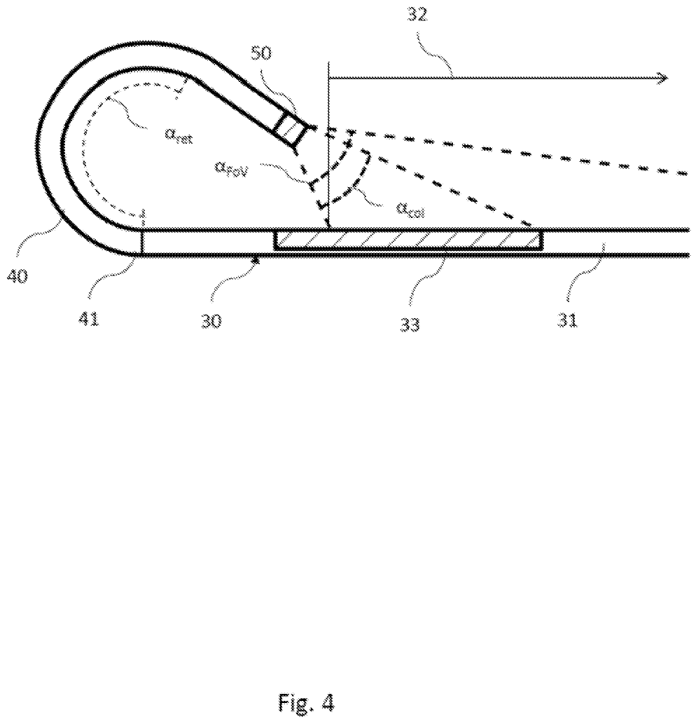

FIG. 1 illustrates an endoscope 1 which is disposable and not intended to be cleaned and reused. The endoscope 1 comprises a distal tip part 50, a handle 20, an insertion tube 30, and a bending section 40. The handle 20 includes a handle housing 21 for gripping and a control mechanism 22 in the form of a control lever. The insertion tube 30 is for insertion into a patient and extends between the handle 20 and a proximal end 41 of the bending section 40. Two steering wires 24a, 24b connect the control lever 22 with the bending section 40. A vision device 42, which in this case is in the form of a camera, and illumination means, such as LED's or light guides/fibre optics 44, are positioned in the distal tip part 50. The insertion tube 30 has an exterior tubular surface 31 facing the surroundings of the endoscope 1. In the present embodiment, the camera has a field of view angle α_{FoV} of about 60°. The control lever 22 is rotatable around a pivot axis to effect a bending movement of the bending section 40 in the plane of the FIGS. 1 and 3-4 by tensioning either of the steering wires 24a, 24b so as to bring the bending section 40 from the first state in which the bending section 40 is unbent and resting as shown in FIG. 1 to a retroflexion state as shown in FIGS. 3-4. The camera is in signal communication with a circuit (not shown) of the handle 20 via data and power cables (not shown). The bending section 40 comprises segments (not shown), hinges (not shown) connecting the segments, and a thin outer sleeve 42 covering the segments and hinges and providing an additional layer of sealing for the connection between the distal tip part 50 and the bending section 40 and also provides a smooth outer surface for the bending section 40 in order to improve the comfort of a patient undergoing endoscopy.

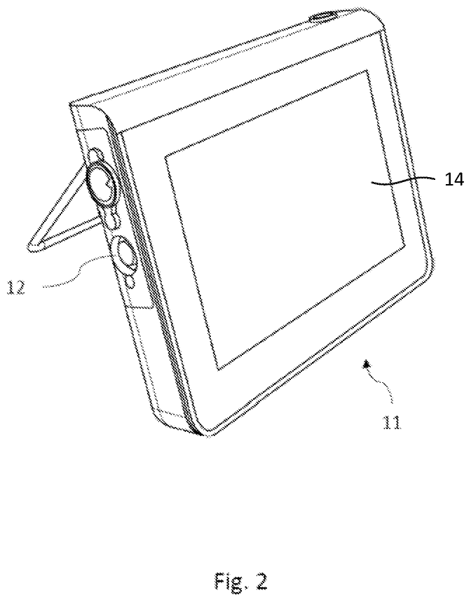

In FIG. 2, a monitor 11 is shown. The monitor 11 comprises a cable socket 12 to which a monitor cable 13 of the endoscope 1 shown in FIG. 1 can be connected to establish signal communication between the camera of the endoscope 1 and the monitor 11 via the circuit of the handle 20. The monitor 11 comprises a display unit 14 to display images captured by the endoscope 1. The monitor 11 thus allows an operator to view images captured by the camera.

As mentioned, FIG. 3 shows the endoscope 1 in the retroflexion state and FIG. 4 shows a more detailed view of the distal end of the endoscope 1 in the retroflexion state. In the retroflexion state, the bending section 40 is bent to a retroflexion angle αᵣₑₜ of about 210° by manipulation of the control lever 22 to an extreme position, i.e. either fully up or fully down, to cause maximum tensioning of one of the steering wires 24a, 24b (FIG. 1) so a first section 32 of the exterior surface 31 enters the field of view (FoV) of the camera. The first section 32 comprises a coloured area 33 covering approximately a distal third of the first section 32. In the retroflexion state, the field of view of the camera includes a view cone with an angle α_{col} of about 45° in which the visible portion of the exterior surface 31 is fully covered by the coloured area 33. This ensures that in the retroflexion state, the majority of the field of view of the camera and in particular the part of the exterior surface 31 closest to the camera is occupied by the coloured area 33 instead of the non-coloured remainder of the exterior surface 31. The coloured area 33 is configured to reduce over- or underexposure of the camera by being of a grey colour with L* between 15 to 75, e.g. 50, a* between negative 10 to positive 10, e.g. 0, and b* between negative 10 to positive 10, e.g. 0, as measured by the CIE L*a*b* colour code system. The exterior surface 31 further comprises a plurality of, e.g. eight, depth marks 34 spaced equidistantly along the extent of the insertion tube 30. Further, a quality inspection mark 35 may be provided, e.g. at a proximal end of the exterior surface 31, indicating that the endoscope 1 has passed quality inspection. The depth marks 34 and/or the quality inspection mark 35 are preferably of the same colour as the coloured area 33.

The coloured area 33, the depth marks 34, and the quality inspection mark 35 are provided by the following method.

Firstly, the insertion tube 30, which is configured for insertion into a patient and which includes the exterior surface 31 facing the surroundings is provided. Then an area of the exterior surface 31, which is visible within a field of view of the camera of the endoscope when the insertion tube 30 forms part of the endoscope 1 and the bending section 40 is in the retroflexion state, is located. A laser then marks the area in the grey colour, thereby providing the coloured area 33 on the insertion tube 30. The grey colour has an L* between 15 to 75, e.g. 50, a* between negative 10 to positive 10, e.g. 0, and b* between negative 10 to positive 10, e.g. 0, as measured by the CIE L*a*b* colour code system. The intensity of the grey colour can be adjusted by adjusting the power of the laser to achieve the desired greyness. Further, the insertion tube 30 can comprise additives to obtain different colours when exposed to light from the laser. The same laser is also used to mark the depth marks 34 and the quality inspection mark 35.

### LIST OF REFERENCES

The following is a list of reference numerals used throughout this disclosure. In case of any doubt, the reference numerals of the following list apply.
1 endoscope
11 monitor
12 cable socket
13 monitor cable
14 display unit
20 handle
21 handle housing
22 control lever
24a steering wire
24b steering wire
30 insertion tube
31 exterior surface
32 first section
33 coloured area
34 depth mark
35 quality inspection mark
40 bending section
41 proximal end
42 sleeve
44 vision device
46 illumination source
50 distal tip part
αᵣₑₜ retroflex angle
α_{FoV} field of view angle
α_{col} field of view angle of the coloured area

1. An endoscope capable of performing a retroflexion manoeuvre, comprising:
a handle having a control mechanism,
a distal tip part having a vision device with a field of view, and an illumination device for providing illumination for the vision device,
a bending section having a first state, a retroflexion state, a proximal end and a distal end connected to the distal tip part,
an insertion tube extending from the handle to the proximal end of the bending section and including an exterior surface facing the surroundings of the endoscope, and
at least one steering wire connecting the control mechanism with the bending section so that manipulation of the control mechanism causes the endoscope to perform a retroflexion manoeuvre by bending the bending section from the first state to the retroflexion state,
wherein the exterior surface of the insertion tube includes a coloured area, of a colour configured to reduce over- or underexposure of the vision device when the bending section of the endoscope is in the retroflexion state.

2. An endoscope according to claim 1, wherein the coloured area is visible within the field of view of the vision device in the retroflexion state of the bending section.

3. An endoscope according to claim 1, wherein the field of view of the vision device includes a view cone with a view cone angle (α_{col}) of at least 40° encompassing the coloured area within the field of view when the endoscope is in the retroflexion state.

4. An endoscope according to claim 1, wherein a majority of the exterior surface of the insertion tube comprises a colour, and wherein the colour of the coloured area is different from the colour of the majority of the exterior surface of the insertion tube.

5. An endoscope according to claim 1, wherein the colour of the coloured area is a grey colour.

6. An endoscope according to claim 1, wherein a first section of the exterior surface is visible within the field of view of the vision device in the retroflexion state of the bending section, wherein the first section comprises a first section area, and wherein the coloured area forms part of the first section and covers at least 20% of the first section area.

7. An endoscope according to claim 6, wherein the first section has a proximal portion and a distal portion, the coloured area covering the distal portion of the first section, the distal portion having a longitudinal extent of at most 30% relative to a longitudinal extent of the exterior surface of the insertion tube.

8. An endoscope according to claim 1, wherein a bending angle of the bending section between the first state and the retroflexion state is between and including 200 and 220 degrees.

9. An endoscope according to claim 1, wherein the insertion tube comprises a plurality of depth marks, spaced along a longitudinal extent of the insertion tube, the depth marks being of the same colour as the coloured area.

10. An endoscope vision system comprising an endoscope according to claim 1 and a monitor, and wherein the endoscope is connectable to the monitor and the monitor includes a display unit configured for displaying images captured by the vision device of the endoscope.

11. A method for marking an area of an endoscope part for an endoscope capable of performing a retroflexion manoeuvre, the endoscope comprising:
a handle having a control mechanism;
a distal tip part having a vision device with a field of view and an illumination device for providing illumination for the vision device;
a bending section having a first state, a retroflexion state, a proximal end, and a distal end connected to the distal tip part;
an insertion tube extending from the handle to the proximal end of the bending section and including an exterior surface facing the surroundings of the endoscope; and
at least one steering wire connecting the control mechanism with the bending section so that manipulation of the control mechanism causes the endoscope to perform a retroflexion manoeuvre by bending the bending section from the first state to the retroflexion state;
wherein the method comprises the steps of:
providing the insertion tube configured for insertion into a patient and including the exterior surface facing the surroundings,
locating an area of the exterior surface of the insertion tube being visible within a field of view of the vision device of the distal tip part of the endoscope when the insertion tube forms part of the endoscope and the bending section is in the retroflexion state, and
marking the area in a colour configured to reduce over- or underexposure of the vision device of the distal tip part of the endoscope, thereby providing a coloured area on the exterior surface of the insertion tube.

12. A method according to claim 11, wherein the insertion tube comprises one or more additives configured for being activated in a laser marking process.

13. A method according to claim 11, further comprising marking the coloured area with a laser in a laser marking process.

14. A method according to claim 13, further comprising a step of:
marking two or more depth marks along a longitudinal extent of the insertion tube.

15. A method according to claim 14, further comprising a step of marking a quality inspection mark on the insertion tube indicating that the endoscope has passed quality inspection.

16. A method according to claim 15, wherein the coloured area, the two or more depth marks, and the quality inspection mark are marked using the same laser marking process, either sequentially by using the same laser or simultaneously by using different lasers.

17. An endoscope according to claim 1, wherein an area visible within the field of view of the vision device in the retroflexion state includes the coloured area, wherein the coloured area covers at least 20% of the area, and wherein a majority of the exterior surface has a colour different than the colour of the coloured area.

18. An endoscope according to claim 17, wherein a bending angle of the bending section between the first state and the retroflexion state is between and including 200 and 220 degrees, and wherein the colour of the coloured area is grey.

19. An endoscope according to claim 6, wherein the first section has a proximal portion and a distal portion having a longitudinal extent of at least 20% relative to a longitudinal extent of the first section, and wherein the coloured area covers the distal portion of the first section.

20. An endoscope according to claim 19, wherein a bending angle of the bending section between the first state and the retroflexion state is between and including 200 and 220 degrees, and wherein the colour of the coloured area is grey.

21. The endoscope of claim 1, wherein the coloured area comprises a surface marked with a laser marking process to reduce a reflectivity of the surface.

22. The endoscope of claim 21, wherein the surface marked with the laser marking process is at least partially melted to reduce the reflectivity of the surface.

23. The endoscope of claim 22, wherein the tube comprises an activatable additive configured to change from an original color to a gray color responsive to exposure to a laser beam, the gray color and the reduced reflectivity of the surface improving an image quality of an image generated by the vision device.

24. The endoscope of claim 21, wherein the vision device is configured to generate images and to change exposure levels to reduce the over- or underexposure in the images, and wherein the marked surface causes the vision device to produce the images with improved quality relative to the surface without said marking.

25. The endoscope of claim 1, wherein the insertion tube comprises an activatable additive configured to change color responsive to exposure to a laser beam.

26. The endoscope of claim 1, wherein an area visible within the field of view of the vision device in the retroflexion state, wherein a retroflexion state angle is 210°, includes a proximal portion and a distal portion, wherein the coloured area covers at least 80% of the area and all of the distal portion, wherein the distal portion includes at least 40% of the area, and wherein a majority of the exterior surface has a colour different than the colour of the coloured area.
